(19) United States
(12) Patent Application Publication
(10) Pub. No.: US 2023/0263743 A1
(43) Pub. Date: Aug. 24, 2023

(54) NANOPARTICLE COMPLEX WITH DEFINED SIZES

(71) Applicant: Suntec Medical, Inc., Walnut, CA (US)

(72) Inventors: Chun-Ting Cheng, Taipei (TW); Cheng-Ying Hsieh, Nantou (TW); Chi-Feng Lin, Taipei (TW); Kuan-Yu Lin, New Taipei (TW); Chao-Ming Su, New Taipei (TW); Pauline Ying Lau, Walnut, CA (US)

(21) Appl. No.: 18/311,114

(22) Filed: May 2, 2023

Related U.S. Application Data
(63) Continuation of application No. PCT/US2021/072307, filed on Nov. 9, 2021.
(60) Provisional application No. 63/112,420, filed on Nov. 11, 2020.

(51) Int. Cl. A61K 9/51; C07K 16/32; C07K 16/28; C07K 16/22; C07K 16/18; A61K 38/20; A61K 38/21; A61K 38/19

(52) U.S. Cl. CPC A61K 9/5146; C07K 16/32; C07K 16/2881; C07K 16/2863; C07K 16/22; C07K 16/18; A61K 38/208; A61K 38/2013; A61K 38/204; A61K 38/2086; A61K 38/20; A61K 38/212; A61K 38/217; A61K 38/191; A61K 9/5123; A61K 9/5192

(57) ABSTRACT

The present invention is directed to nanoparticles comprising: (a) an inner core comprising oligomeric (−)-epigallocatechin gallate (OEGCG), (b) an outer core comprising a polyethylene glycol-epigallocatechin gallate conjugate (PEG-EGCG), and (c) a protein molecule of a drug substance encapsulated in the inner core; wherein at least 70% of the nanoparticles have a diameter between 50-300 nm. The present invention also provides a process for preparing the nanoparticles. The present invention further provides a method of treating cancer by administering to a subject in need thereof the nanoparticles of the present invention, in an amount effective to treat cancer.

This application is a continuation of PCT/US2021/072307, filed Nov. 9, 2021; which claims the benefit of U.S. Provisional Application No. 63,112,420, filed Nov. 11, 2020. The contents of the above-identified applications are incorporated herein by reference in their entirety.

### FIELD OF THE INVENTION

The present invention relates to nanoparticles comprising: (a) an inner core comprising oligomeric (−)-epigallocatechin gallate (OEGCG), (b) an outer core comprising a PEG-EGCG conjugate, and (c) a drug molecule encapsulated in the inner core; wherein at least 70% of the nanoparticles have a diameter between 50-300 nm. The present invention also relates to a process making the nanoparticles.

### BACKGROUND OF THE INVENTION

Green tea catechins have health benefits of prevention of cardiovascular diseases and cancers. Among tea catechins, (−)-epigallalocatechin-3-gallate (EGCG) is the most abundant and it plays a major role in the beneficial effects of green tea. EGCG possesses antioxidant, anti-inflammatory, and immune modulation effects. EGCG has also been shown to effectively inhibit tumor growth and metastasis by targeting multiple signal transduction pathways essential for cancer cell survival.

Despite these desirable activities, clinical applications of EGCG have been limited by its poor stability and low oral bioavailability. For instance, EGCG is unstable and easily decomposed under physiological environment. As a result, plasma concentrations of EGCG required to achieve a desired therapeutic effect cannot be reached following oral administration.

There are three major challenges to the treatment of cancer, a complicated disease with multiple signaling pathways. First, cancers are erupted from a person's immune dysfunctions. Immune modulation to restore host immune function is critical for long term treatment resolution. Second, a single therapeutic agent can only modify one disease pathway and has limited efficacy, drug-resistance, and non-response. Cancer cells can escape from a single agent treatment through alternative signaling pathways. Third, drug toxicity and non-effective dosing to target tissue are common challenges to cancer therapies because tumor size is a small fraction of body size. Only a small fraction of the drug administered reaches targeted tissue, and the majority of the drug enters non-targeted normal tissues which causes low efficacy to targeted tissue and high toxicity to normal tissues.

The molecular size of a drug determines how much of the drug selectively goes to a target tissue (e.g., inflammation and fast-growing tissue) versus unintended other tissues (Chem Soc Rev Oct. 28, 2019; 48(21):5381-5407). Normal, unintended healthy tissues have blood vessel openings of less than 10 nm in general. Tumor tissues have gaps of about 300-1000 nm in the vessel wall. Other inflammation tissues, such as autoimmune disease organs, have various gap size, generally also greater than 300 nm.

There is a need for a pharmaceutical composition and a drug delivery system that overcomes the challenges described above and effectively enters target tissue without potential toxicity.

### BRIEF DESCRIPTION OF THE DRAWING

FIG. 1 shows a nanoparticle micelle composition of the present invention, in which a drug molecule is encapsulated within the micelle, and the micelle comprises PEG-EGCG conjugate and oligomeric EGCG (OEGCG).

FIG. 2 shows the nanoparticle size distribution of a nanoparticle micelle composition of the present invention comprising trastuzumab.

FIG. 3 shows the nanoparticle size distribution of a nanoparticle micelle composition of the present invention comprising IL-12.

FIG. 4 shows the nanoparticle size distribution of a nanoparticle micelle composition of the present invention comprising anti-CD3.

FIG. 5 shows the nanoparticle size distribution of a nanoparticle micelle composition of the present invention comprising IFN (interferon)-α.

FIGS. 6A and 6B show comparison of trastuzumab nanoparticle micelle compositions prepared with different trastuzumab to OEGCG molar ratios.

FIGS. 7A and 7B show trastuzumab nanoparticle micelle compositions prepared with (FIG. 7B) or without (FIG. 7A)10K molecular weight cut-off ultrafiltration.

FIGS. 8A and 8B show comparison of trastuzumab nanoparticle micelle compositions prepared with (FIG. 8B) or without (FIG. 7A) 0.22 μm filtration.

FIG. 9 shows comparison of trastuzumab nanoparticle micelle compositions prepared by stepwise freezing (invention), continuous freezing (comparison), or one-step freezing (comparison).

FIG. 10 shows tumor volume vs. time in mice after treatment by control, trastuzumab, and trastuzumab nanoparticle micelle composition.

FIG. 11 shows the nanoparticle size distribution of a nanoparticle micelle composition of the present invention comprising anti-CD71.

FIG. 12 shows the nanoparticle size distribution of a nanoparticle micelle composition of the present invention comprising anti-epidermal growth factor receptor (EGFR).

FIG. 13 shows the nanoparticle size distribution of a nanoparticle micelle composition of the present invention comprising anti-Tau.

FIG. 14 shows the nanoparticle size distribution of a nanoparticle micelle composition of the present invention comprising anti-vascular endothelial growth factor (VEGF).

FIG. 15 shows the nanoparticle size distribution of a nanoparticle micelle composition of the present invention comprising IFN-γ.

FIG. 16 shows the nanoparticle size distribution of a nanoparticle micelle composition of the present invention comprising interleukin (IL)-2.

FIG. 17 shows the nanoparticle size distribution of a nanoparticle micelle composition of the present invention comprising IL-6.

FIG. 18 shows the nanoparticle size distribution of a nanoparticle micelle composition of the present invention comprising IL-15.

FIG. 19 shows the nanoparticle size distribution of a nanoparticle micelle composition of the present invention comprising IL-21.

FIG. 20 shows the nanoparticle size distribution of a nanoparticle micelle composition of the present invention comprising tumor necrosis factor-related apoptosis-inducing ligand (TRAIL).

FIG. 21 shows the nanoparticle size distribution of a nanoparticle micelle composition of the present invention comprising bovine serum albumin (BSA).

### DETAILED DESCRIPTION OF THE INVENTION

### Definition

The term “about” is defined as +10%, preferably +5%, of the recited value.

The term “cytokines” refer to small proteins (-5-70 kDa) important in cell signaling. Cytokines have been shown to be involved in autocrine, paracrine, and endocrine signaling as immunomodulating agents. Cytokines include interferons, interleukins, lymphokines, tumor necrosis factors, and chemokines.

The term “epigallocatechin gallate” refers to an ester of epigallocatechin and gallic acid, and is used interchangeably with “epigallocatechin-3-gallate” or EGCG.

The term “nanoparticles” refers to particles with a diameter below 1p m and between 1-999 nm.

The term “oligomeric EGCG” (OEGCG) refers to 3-20 monomers of EGCG that are covalently linked. OEGCG preferably contains 4 to 12 monomers of EGCG. The structure of OEGCG is shown in WO2006/124000. OEGCG, for example, can be synthesized according to WO2006/124000.

The term “polyethylene glycol-epigallocatechin gallate conjugate” or “PEG-EGCG refers to polyethylene glycol (PEG) conjugated to one or two molecules of EGCG. The term “PEG-EGCG” refer to both PEG-mEGCG conjugate (monomeric EGCG) and PEG-dEGCG (dimeric EGCG) conjugate. PEG-EGCG, for example, can be prepared by conjugating aldehyde-terminated PEG to EGCG by attachment of the polyethylene glycol via reaction of the free aldehyde group with the C6 and/or C8 position of the A ring of EGCG. See WO2006/124000 and WO2009/054813.

### Nanoparticle Composition

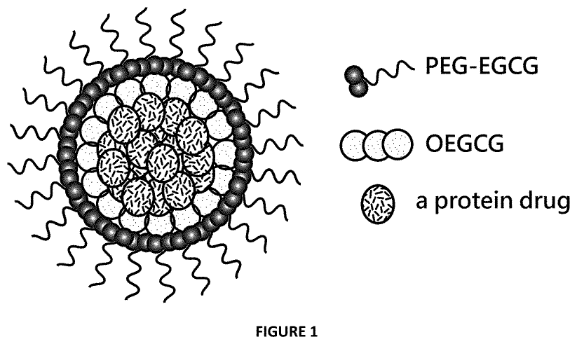

The present invention provides a nanoparticle micelle composition in which a drug molecule is encapsulated within the micelle, and the micelle comprises PEG-EGCG conjugate in an outer core and oligomeric EGCG (OEGCG) in an inner core (see FIG. 1). The nanoparticle micelle composition has a defined and narrow size distribution in that at least 70% of the nanoparticles have a diameter between 50-300 nm, and the size distribution of the nanoparticles has only one major peak containing more than 90% of all the nanoparticles.

The present nanoparticle micelle composition comprises three active ingredients, which are complementary in function to form a “multiple targeted combination therapy” to tackle both immune response and signaling pathways by its backbone components (OEGCG/PEG-EGCG), and additional signaling pathways by a selected protein drug molecule effective in treating complicated diseases. Each nanoparticle is a fixed-dose combination drug with the three active ingredients at fixed molar ratio.

The present composition treats diseases via multiple signal pathways. It provides cancer therapy with enhanced efficacy (tumor reduction rate) and increased patient response rate (number of patients who respond to the treatment).

The nanoparticle micelle composition of the present invention has a majority of the particles in the size of 50-300 nm. The defined nanoparticle size of 50-300 nm allows for preferential distribution of the three active ingredients to the tumor, which diminishes their penetration to other non-tumor tissues. If the particle size is smaller than 50 nm, there is a higher risk that the particle will be distributed to normal tissues and cause cytotoxicity. If the particle size is greater than 300 nm, it may cause excess uptake by reticuloendothelial (RE) system and resulting in side effects. The present composition has more than 70% of the particles in the size range of 50-300 nm, which ensures the three active ingredients enter the tumor preferentially over normal tissue and RE system.

The nanoparticle micelle composition of the present invention has a narrow particle size distribution in that it has only one major peak that contains more than 90% of all the nanoparticles. It is important to have a therapeutic composition having only one peak of particle size distribution, instead of several peaks or multiple peaks. If a therapeutic composition has more than a single molecular size, it can cause severe variations in therapeutic efficacy, patient response rate, and adverse effects (toxicity).

The nanocomplex of the present invention contains the first two active ingredients, OEGCG and PEG-EGCG, which are immune modulators and signaling regulators, in the backbone of the micelle composition. They are derivatives of EGCG, which is a strong immune modulator and regulates a wide spectrum of disease signaling pathways. For example, EGCG activates tumor-targeting CD8+ T cells, and suppresses anti-PD-L1 expression in cancers. EGCG regulates both innate and adaptive immunity in autoimmune diseases. However, the bioavailability of EGCG is low and EGCG is not stable. The present nanocomplex composition overcomes the bioavailability issue of EGCG by forming a nanocarrier to carry EGCG to the tumor, and overcomes the stability issue of EGCG by forming OEGCG and PEG-EGCG, which effectively enables EGCG as highly effective therapeutic agents.

The nanocomplex of the present invention further contains a third active ingredient, which is a drug molecule encapsulated in the nanoparticles. The drug molecule is preferably a protein drug, and includes, but is not limited to, cytokines and antibodies. Cytokines include, but are not limited to, IL-2, IL-6, IL-7, IL-10, IL-12, IL-15, IL-17, IL-21, TARIL, IGF1, GLP-1, IFN-α, IFN-β, IFN-γ, CCL5, CXCL9, CXCL10, CXCL11, CX3CL1, and recombinant cytokine products. Antibodies include, but are not limited to, monoclonal antibody, polyclonal antibody, antibody-drug-conjugate, and bispecific antibody. Preferred antibodies for the present invention are monoclonal antibodies. Antibodies suitable for the present invention include anti-PD-1 antibody, anti-PD-L1 antibody, anti-CTLA-4 antibody, anti-LAG3 antibody, anti-TIGIT antibody, anti-TIM3 antibody, anti-HER2 antibody, anti-HER3 antibody, anti-HGFR antibody, anti-EGFR antibody, anti-EpCAM, anti-FOLR1 antibody, anti-c-Met antibody, anti-GD2 ganglioside antibody, anti-GD3 ganglioside, anti-VEGFR1 antibody, anti-VEGF antibody, anti-TGF-β antibody, anti-TNF-α antibody, anti-IGF-1R antibody, anti-IL-4 antibody, anti-IL-10 antibody, anti-IL-13 antibody, anti-CD3 antibody, anti-CD4 antibody, anti-CD40 antibody, anti-CD40L antibody, anti-CD43 antibody, anti-CD19 antibody, anti-CD27 antibody, anti-CD70 antibody, anti-CD71 antibody, anti-CD28 antibody, anti-CD38 antibody, anti-CD20 antibody, anti-B7-H3 antibody, anti-B7-H4 antibody, anti-DR5 antibody, anti-MUC1 antibody, anti-Tau antibody, anti-0 amyloid antibody, abagovomab, abituzumab, adalimumab, aducanumab, alemtuzumab, amatuximab, amivantamab, anifrolumab, atezolizumab, avelumab, bapineuzumab, basiliximab, belimumab, benralizumab, besilesomab, bevacizumab, bezlotoxumab, blinatumomab, brazikumab, brontictuzumab, cabiralizumab, camrelizumab, carlumab, carotuximab, catumaxomab, cedelizumab, cetrelimab, cetuximab, cibisatamab, crenezumab, cusatuzumab, daclizumab, daclizumab, dalotuzumab, daratumumab, detumomab, dinutuximab, drozitumab, duligotuzumab, dupilumab, durvalumab, ecromeximab, emibetuzumab, epcoritamab, epratuzumab, eptinezumab, erenumab, ertumaxomab, etaracizumab, etesevimab, farletuzumab, fezakinumab, ficlatuzumab, figitumumab, fletikumab, foralumab, fresolimumab, futuximab, ganitumab, gantenerumab, gatipotuzumab, gevokizumab, golimumab, guselkumab, icrucumab, igovomab, imalumab, imgatuzumab, inebilizumab, infliximab, intetumumab, ipilimumab, istiratumab, ixekizumab, letolizumab, lexatumumab, lintuzumab, mapatumumab, matuzumab, mavrilimumab, mepolizumab, mogamulizumab, monalizumab, mosunetuzumab, natalizumab, naxitamab, necitumumab, nimotuzumab, nivolumab, ocaratuzumab, ocrelizumab, ofatumumab, olaratumab, olaratumabopicinumab, panitumumab, pembrolizumab, pertuzumab, ponezumab, ramucirumab, ranibizumab, rituximab, samalizumab, sarilumab, secukinumab, sintilimab, solanezumab, teprotumumab, tigatuzumab, tildrakizumab, timigutuzumab, tocilizumab, tomuzotuximab, trastuzumab, ustekinumab, vanucizumab, varisacumab, varlilumab, vedolizumab, vepalimomab, vesencumab, visilizumab, vonlerolizumab, zanolimumab, zatuximab, zenocutuzumab, zolbetuximab, ado-trastuzumab emtansine, anetumab ravtansine, brentuximab vedotin, cantuzumab mertansine, certolizumab pegol, coltuximab ravtansine, depatuxizumab mafodotin, enapotamab vedotin, gemtuzumab ozogamicin, glembatumumab vedotin, iladatuzumab vedotin, inatuzumab vedotin, indatuximab ravtansine, indusatumab vedotin, lifastuzumab vedotin, lilotomab satetraxetan, lorvotuzumab mertansine, losatuxizumab vedotin, lulizumab pegol, mirvetuximab soravtansine, naratuximab emtansine, notuzumab ozogamicin, polatuzumab vedotin-piiq, rovalpituzumab tesirine, sacituzumab govitecan, samrotamab vedotin, telisotuzumab vedotin, trastuzumab deruxtecan, and tucotuzumab celmoleukin. Antibodies also include antibody fragments that are capable of binding to their corresponding antigens, such as Fab, (Fab)₂, or single-chain antibodies.

Nanoparticles with sizes of 10-50 nm tend to enter both normal and target tissues from blood circulation.

Nanoparticles with sizes 50-300 nm preferentially go to tumor or inflammation tissues. Large nanoparticles (500-999 nm) or micron-size (1000-5000 nm) particles, due to aggregation of smaller nanoparticles, may lead to toxicity because large nanoparticles are often efficiently taken up by the reticuloendothelial system (RES), also known as the mononuclear phagocytic system (MPS) located in the liver, lungs, and bone marrow. This may reduce the efficacy of nanoparticle drugs at desired disease lesions and leads to potential toxicity.

The inventors have discovered a nanoparticle micelle composition comprising EGCG and a drug agent for the target delivery to target tissue, with at least 70% of the nanoparticles having a diameter between 50-300 nm, and the size distribution of the nanoparticles only has one major peak that contains more than 90% of all the particles. The inventors have also discovered a process for preparing such nanoparticle composition.

The present invention is directed to a nanoparticle composition comprising nanoparticles having: (a) an inner core comprising oligomeric (−)-epigallocatechin gallate (OEGCG), (b) an outer core comprising a PEG-EGCG conjugate, and (c) a drug molecule encapsulated in the inner core; wherein at least 70% of the nanoparticles have a diameter between 50-300 nm, and the size distribution of the nanoparticles only has one major peak that contains more than 90% of all the particles.

The structure of the nanoparticles of the present invention is shown in FIG. 1.

In one embodiment, at least 80%, or at least 85%, or at least 90%, or at least 95% of the nanoparticles have a diameter between 50-300 nm.

In one embodiment, the median nanoparticle diameter in the nanoparticle composition is between 50 to 250 nm, 50 to 200 nm, 80 to 200 nm, 100 to 200 nm, or 50 to 150 nm.

In one embodiment, the size distribution of the nanoparticles only shows one major and narrow peak that contains more than 80%, more than 85%, more than 90%, more than 95%, or more than 98% of all the particles.

In one embodiment, the protein drug is trastuzumab, and at least 80% or at least 90% of the nanoparticles in the nanoparticle composition have a diameter between 50-300 nm. The median nanoparticle diameter in the nanoparticle composition is between 60 to 200 nm.

In one embodiment, the protein drug is IL-12, and at least 75% of the nanoparticles have a diameter between 50-300 nm. The median diameter of the nanoparticles is between 60 to 200 nm.

In one embodiment, the protein drug is anti-CD3, and at least 80% or at least 90% of the nanoparticles have a diameter between 50-300 nm. The median nanoparticle diameter is between 60 to 200 nm.

In one embodiment, the protein drug is IFN-α, and at least 80% or at least 90% of the nanoparticles have a diameter between 50-300 nm. The median nanoparticle diameter is between 60 to 200 nm.

The nanoparticle composition of the present invention has a majority of particles size of 50-300 nm in diameter with OEGCG, PEG-EGCG, and a drug molecule held together by hydrophobic interactions. It is stable in a hydrophilic environment, such as blood circulation, and dissociates in a hydrophobic environment, such as a tumor tissue. It can selectively diffuse from blood vessels to surrounding tissue with leaky vessels due to inflammation and other hyperactivities, such as rapid, uncontrolled tumor growth. Due to its size, it is restricted from entering normal tissues with less leaky vessels. Once the nanoparticle complex enters tissue which is hydrophobic, it dissociates and frees its active components OEGCG, PEG-EGCG, and the drug molecule in the nanocomplex. The free active components regain their bioactivities in cancer retardation. The active components in the nanoparticles have a longer circulation half-life and act as a slow-release mechanism which further lowers the drug dosage requirement. Consequently, any adverse effects to normal tissues are further diminished.

### Process for Preparing the Nanoparticle Composition

The present invention is also directed to a process for preparing nanoparticle composition of a fixed-dose combination drug. The process is optimized so only the nanometer-size particles with at least 70% of the particles having a diameter between 50-300 nm are produced.

The process comprises the steps of: (a) mixing a drug molecule with OEGCG and PEG-EGCG in an aqueous solution; (b) filtering the mixture through a membrane with a molecular weight cut-off of 8,000-300,000 daltons to remove small molecular weight molecules and retain large molecular weight molecules; and (c) filtering the large molecular weight molecules through 0.2-0.3 μm membrane and collecting the filtrate.

The present process optionally further comprises a lyophilization step (d) after step (c). Step (d): lyophilizing the filtrate by stepwise freezing at (i) about 0-5° C., (ii) about −20 to −30° C., and (iii) at about −60 to −100° C., and then drying.

In step (a), the drug molecule is dissolved in an aqueous solvent, such as phosphate-buffer saline, saline, water, bicarbonate buffer, oxyhemoglobin buffer, bis-tris alkane, Tris-HCl, HEPES, histidine buffer, NP-40, RIPA (radioimmunoprecipitation assay buffer), tricine, TES, TAPS, TAPSO, Bicine, MOPS, PIPES, cacodylate, or MES. Preferred solvents are phosphate-buffer saline, saline, or water. The protein drug concentration is in general 0.01-50 mg/ml, preferred 0.05-10 mg/ml, and more preferred 0.1-5 mg/ml.

OEGCG, PEG-EGCG, and optionally EGCG, are dissolved in ketones, acetonitrile, alcohols, aldehydes, ethers, acetates, sulfoxides, benzenes, organic acids, amides, aqueous buffers, and any combination thereof. Preferred solvents are alcohols, acetonitrile, sulfoxides, amides, and any combination thereof. The OEGCG/EGCG and PEG-EGCG concentrations are in general independently 0.001-10 mg/ml, preferred 0.005-1 mg/ml, or 0.1-5 mg/ml.

It is important that OEGCG is in molar excess of the drug agent. In general, the molar ratio of the EGCG in OEGCG to the drug molecule is between 1-500 to 1, 2-500 to 1, 3-500 to 1, or 5-500 to 1, preferably 3-100 to 1, 5-100 to 1, or 10-50 to 1. The molar ratio is calculated by the number of moles of monomer EGCG in OEGCG to the number of moles of the drug molecule. The molar excess of EGCG ensures most or all drug agents are encapsulated by the OEGCG molecules. Unencapsulated drug agents, which would not be selectively distributed to target tissue and would cause lower efficacy and safety issues, are avoided by controlling the molar ratio of OEGCG to protein in the present process.

The drug agent, OEGCG, and PEG-EGCG are mixed between 1 minute to 2 days, preferably 1 minute to 12 hours, at a temperature between about 0° C. to 60° C., preferably 0° C. to 45° C., or 0° C. to 37° C.

In step (b), the above mixture is filtered through a membrane with a molecular weight cut-off between 8,000-300,000 daltons, preferably between 8,000-200,000 daltons, 8,000-150,000 daltons, or 8,000-12,000 daltons, to remove small molecular weight molecules and retain large molecular weight molecules. The ultrafiltration membrane material is selected from the group consisting of cellulose (and its derivatives), polyethersulfone (PES), polytetrafluoroethylene (PTFE), nylon, polyvinylidene fluoride or polyvinylidene difluoride (PVDF), and polypropylene (PP); preferably cellulose (and its derivatives), PTFE, and PVDF.

The mixture is optionally diluted in an aqueous solvent such as those described above in step (a) before ultrafiltration.

The ultrafiltration step (b) removes unwanted impurities of small molecular weight, such as unreacted OEGCG or EGCG, or reaction by-products. These impurities may reduce drug efficacy and safety. The excess of unreacted OEGCG or EGCG may also lead to aggregation of the individual nanoparticles to about 1000 nm size particles, which would reduce efficacy and cause potential toxicity.

In step (c), the retained large molecular weight molecules are filtered through a membrane having a pore size of about 0.2-0.3 μm, such as 0.22 μm, and the filtrate is collected. This is to remove unwanted impurities of large molecular sizes, such as mega-aggregates. These aggregates may be excreted from entering tissues due to its mega size. These aggregates reduce overall efficacy/safety and have a higher chance of inducing immunogenicity to the patients. Large size nanoparticles are also easier to be taken up by RES in the liver, lungs, and more undesired organs.

The membrane material of step (c) is selected from the group consisting of cellulose (and its derivatives), PES, PTFE, nylon, PVDF, and PP; preferably cellulose (and its derivatives), PES, and PP.

In one embodiment, the steps (b) and (c) are repeated at least one time, for example, repeated 1, 2, 3, or 4 times before step (d), to effectively remove unwanted small molecule impurities and large aggregates.

After step (c), the filtrate is stored at 2-8° C., and is stable for at least 100 days.

The present process optionally further comprises a lyophilization step (d) after step (c) to provide a long-term stability of the nanoparticle composition.

In step (d), the filtrate collected after filtration through 0.2-0.3 μm membrane is lyophilized by first stepwise freezing the filtrate at (i) about 0-5° C., for example, for about 1-3 hours, (ii) about −25° C. to −30° C., for example, for about 1-3 hours, then freezing at (iii) −60° C. to −100° C. or −70° C. to −100° C., for example, for at least 8 hours.

After freezing, the material is lyophilized for 1 to 7 days.

Freezing and lyophilization often cause nanoparticles to form complexes and aggregates. These large particles may be too big to penetrate blood vessels and enter tissue environments. Consequently, efficacy and safety are lower, and immunogenicity may increase. To avoid these changes caused by lyophilization, the present process uses a stepwise freezing process, instead of a continuously freezing process (lowering temperature gradually and continuously during freezing), to preserve the nanoparticle size during lyophilization.

### Pharmaceutical Compositions

The present invention provides pharmaceutical compositions comprising the nanoparticle composition of the present invention and optionally one or more pharmaceutically acceptable carriers. The nanoparticle composition in a pharmaceutical composition in general is about 1-90%, preferably 20-90%, or 30-80% for a tablet, powder, or parenteral formulation. The nanoparticle composition in a pharmaceutical composition in general is 1-100%, preferably 20-100%, 50-100%, or 70-100% for a capsule formulation. The nanoparticle composition in a pharmaceutical composition in general is 1-50%, 5-50%, or 10-40% for a liquid suspension formulation.

In one embodiment, the pharmaceutical composition can be in a dosage form such as tablets, capsules, granules, fine granules, powders, suspension, patch, parenteral, injectable, or the like. The above pharmaceutical compositions can be prepared by conventional methods.

Pharmaceutically acceptable carriers, which are inactive ingredients, can be selected by those skilled in the art using conventional criteria. The pharmaceutically acceptable carriers may contain ingredients that include, but are not limited to, saline and aqueous electrolyte solutions; ionic and nonionic osmotic agents, such as sodium chloride, potassium chloride, glycerol, and dextrose; pH adjusters and buffers, such as salts of hydroxide, phosphate, citrate, acetate, borate, and trolamine; antioxidants, such as salts, acids, and/or bases of bisulfite, sulfite, metabisulfite, thiosulfite, ascorbic acid, acetyl cysteine, cysteine, glutathione, butylated hydroxyanisole, butylated hydroxytoluene, tocopherols, and ascorbyl palmitate; surfactants, such as lecithin and phospholipids, including, but not limited to, phosphatidylcholine, phosphatidylethanolamine and phosphatidyl inositol; poloxamers and poloxamines; polysorbates, such as polysorbate 80, polysorbate 60, and polysorbate 20; polyethers, such as polyethylene glycols and polypropylene glycols; polyvinyls, such as polyvinyl alcohol and polyvinylpyrrolidone (PVP, povidone); cellulose derivatives, such as methylcellulose, hydroxypropyl cellulose, hydroxyethyl cellulose, carboxymethyl cellulose, and hydroxypropyl methylcellulose and their salts; petroleum derivatives, such as mineral oil and white petrolatum; fats, such as lanolin, peanut oil, palm oil, and soybean oil; mono-, di-, and triglycerides; polysaccharides, such as dextrans; and glycosaminoglycans, such as sodium hyaluronate. Such pharmaceutically acceptable carriers may be preserved against bacterial contamination using well-known preservatives, which include, but are not limited to, benzalkonium chloride, ethylene diamine tetra-acetic acid and its salts, benzethonium chloride, chlorhexidine, chlorobutanol, methylparaben, thimerosal, and phenylethyl alcohol, or may be formulated as a non-preserved formulation for either single or multiple use.

For example, a tablet, capsule, or parenteral formulation of the active compound may contain other excipients that have no bioactivity and no reaction with the active compound. Excipients of a tablet or a capsule may include fillers, binders, lubricants and glidants, disintegrators, wetting agents, and release rate modifiers. Examples of excipients of a tablet or a capsule include, but are not limited to, carboxymethylcellulose, cellulose, ethylcellulose, hydroxypropylmethylcellulose, methylcellulose, karaya gum, starch, tragacanth gum, gelatin, magnesium stearate, titanium dioxide, poly(acrylic acid), and polyvinylpyrrolidone. For example, a tablet formulation may contain inactive ingredients, such as colloidal silicon dioxide, crospovidone, hypromellose, magnesium stearate, microcrystalline cellulose, polyethylene glycol, sodium starch glycolate, and titanium dioxide. A capsule formulation may contain inactive ingredients, such as gelatin, magnesium stearate, and titanium dioxide. A powder oral formulation may contain inactive ingredients, such as silica gel, sodium benzoate, sodium citrate, sucrose, and xanthan gum.

### Method of Use

The present invention is directed to a method of making a combination drug for treating cancer and other diseases. The method comprises the step of administering an effective amount of the nanoparticle composition of the present invention to a subject in need thereof. “An effective amount,” as used herein, is the amount effective to treat a disease by ameliorating the pathological condition or reducing the symptoms of the disease.

The pharmaceutical composition of the present invention can be applied by local administration and systemic administration. Local administration includes topical administration. Systemic administration includes oral, parenteral (such as intravenous, intramuscular, subcutaneous, or rectal), and other systemic routes of administration. In systemic administration, the active compound first reaches plasma and then distributes into target tissues. Parenteral administration, such as intravenous bolus injection or intravenous infusion, and oral administration are preferred routes of administration for the present nanoparticle composition.

In one embodiment, the protein drug in the nanoparticle composition is an anti-HER2 antibody such as trastuzumab, which is approved for treating breast cancer caused by the HER2 receptor pathway. Only 20% of breast cancer patients respond to trastuzumab therapy. The rest (80%) of the breast cancers are caused by mutations of alternative signal-pathways. OEGCG and PEG-EGCG in the present nanoparticle composition can modulate alternative signal pathways of breast cancer to treat the 80% of breast cancer patients who are trastuzumab non-responders. Multi-target immune nanocarrier combination (MINC)-trastuzumab is also suitable for treating bladder cancer, brain cancer, cervical cancer, cholangiocarcinoma, colorectal cancer, esophageal cancer, gallbladder cancer, gastric cancer, head and neck carcinoma, hepatocellular carcinoma, kidney cancer, lung cancer, ovarian cancer, pancreatic cancer, prostate cancer, testicular cancer, and uterine cancer.

In one embodiment, the protein drug in the nanoparticle composition is IL-12, including those on clinical trials such as NHS-IL-12 and rHU-IL-12. IL-12 is known to widely impact key immune cells, including CD4+ T, CD8+ T, B, and NK cells. However, IL-12 is very toxic due to its high impacts on human immune mechanisms. MINC-IL-12 can drastically reduce its toxicity while enhancing its efficacy to allow for the use of IL-12 as a wide spectrum immunotherapy for many cancers. MINC-IL-12 is suitable to treat bladder cancer, brain cancer, breast cancer, cervical cancer, childhood cancer, colorectal cancer, esophageal cancer, head and neck carcinoma, kidney cancer, liver cancer, lung cancer, lymphoma, melanoma, multiple myeloma, ovarian cancer, pancreatic cancer, prostate cancer, sarcoma, skin cancer, stomach cancer, and uterine cancer. MINC-IL12 can be combined with another cancer immunotherapeutic, such as anti-PD-1, anti-PD-L1, anti-CTLA-4, anti-TIGIT, anti-LAG3, or anti-TIM3 to improve therapeutic efficacy.

In one embodiment, the protein drug in the nanoparticle composition is anti-CD3 antibodies including teplizumab, muromonab, otelixizumab, and visilizumab. Type 1 diabetes is an autoimmune disease caused by T cell attacks on pancreatic beta cells. Anti-CD3 is very toxic, with severe adverse responses from a clinical trial. MINC-anti-CD3 can preferentially carry anti-CD3 to pancreatic beta cells and leave low exposure to normal cells, which provides enhanced efficacy and safety. Additionally, EGCG has a function to enhance proliferation of pancreatic beta cells and help to restore its function in insulin secretions. MINC-anti-CD3 is suitable to treat rheumatoid arthritis, inflammatory bowel diseases, psoriasis, and some other autoimmune diseases.

In one embodiment, the protein drug in the nanoparticle composition is alpha-interferon. OEGCG and PEG-EGCG can activate T cells and suppress anti-PD-L1 to help cancer patients better respond to immunotherapies for cancer. IFN-α can also induce IFN-7 production and further suppress tumor growth. Nanoparticles can send active ingredients, OEGCG, PEG-EGCG, and IFN-α, to tumors over normal tissues to enhance efficacy and reduce toxicity of IFN-α. MINC-IFN-α is applicable to most cancer types who need to boost the response to immunotherapies, including bladder cancer, brain cancer, breast cancer, cervical cancer, childhood cancer, colorectal cancer, esophageal cancer, head and neck carcinoma, kidney cancer, liver cancer, lung cancer, lymphoma, melanoma, multiple myeloma, ovarian cancer, pancreatic cancer, prostate cancer, sarcoma, skin cancer, stomach cancer, and uterine cancer.

In one embodiment, the protein drug in the nanoparticle composition is anti-CD71. Anti-CD71 targets CD71, also known as transferrin receptor protein 1, which is a target in cases of human leukemia and lymphoma.

In one embodiment, the protein drug in the nanoparticle composition is anti-EGFR. Anti-EGFR is an epidermal growth factor receptor (EGFR) inhibitor medication used for the treatment of metastatic colorectal cancer, head and neck cancer and more EGFR positive cancers.

In one embodiment, the protein drug in the nanoparticle composition is anti-Tau. Tau protein causes the pathologies and dementias of the nervous system, such as Alzheimer's disease and Parkinson's disease. Anti-Tau can be used for treating nervous system diseases via targeting the tau protein.

In one embodiment, the protein drug in the nanoparticle composition is anti-VEGF. Anti-VEGF is used to block vascular endothelial growth factor for treating certain cancers and age-related macular degeneration.

In one embodiment, the protein drug in the nanoparticle composition is IFN-γ. IFN-γ is a cytokine that is critical for innate and adaptive immunity against viral, some bacterial, and protozoan infections. IFN-γ is also useful for treating cancers.

In one embodiment, the protein drug in the nanoparticle composition is IL-2. IL-2 increases the cell killing activity of both natural killer cells and cytotoxic T cells. IL-2 can be used for the treatment of cancers including malignant melanoma and renal cell cancer.

In one embodiment, the protein drug in the nanoparticle composition is IL-6. IL-6 is an interleukin that acts as both a pro-inflammatory cytokine and an anti-inflammatory myokine.

In one embodiment, the protein drug in the nanoparticle composition is IL-15. IL-15 is a cytokine with a structural similarity to Interleukin-2 (IL-2). IL-15 has been shown to enhance the anti-tumor immunity of CD8+ T cells.

In one embodiment, the protein drug in the nanoparticle composition is IL-21. IL-21 has regulatory effects on cells of the immune system, including natural killer (NK) cells and cytotoxic T cells that can destroy virally infected or cancerous cells.

In one embodiment, the protein drug in the nanoparticle composition is TRAIL. TRAIL is a protein functioning as a ligand that induces the process of cell death called apoptosis.

In one embodiment, the protein drug in the nanoparticle composition is IL-21. IL-21 has anti-tumor effects through continued and increased CD8+ T cell response to achieve enduring anti-tumor immunity.

In one embodiment, a protein is used in the nanoparticle composition, such as BSA. BSA is bovine a serum albumin protein derived from cows. It is often used as a protein concentration standard in lab experiments.

Dosing of the nanoparticle composition is based on the known dosage of the protein drug for treating a particular disease and the subject condition. For example, for treating breast cancer in an adult human, trastuzumab is administered 4-8 mg/kg via IV infusion once weekly for 52 weeks. The effective dose of MINC-trastuzumab is in the same dose range with a less frequent dosing frequency of once every 12 to 16 weeks for 52 weeks.

For treating type 1 diabetes, a 14-day course of anti-CD3 is administered by IV injection at 1 to 20 μg/kg for a 14-day course only once for everyone's life without repeat dosing due to drug toxicity. The effective dose of MINC-anti-CD3 in the same dose range is administered in a 3-5-day course per year and can be dosed repeatedly once every year.

For treating melanoma, interferon-α induction is 20 million IU/m² as an IV infusion, at 5 consecutive days per week for 4 weeks. The effective dose of MINC-interferon-α is in the same dosage range, administered at 1 day per week for 2 weeks to achieve the same efficacy and reduced toxicity.

For treating kidney cancer, IL-12 at 600,000 International Units/kg (0.037 mg/kg) is administered three times a day for a maximum of 14 doses. Following 9 days of rest, the schedule is repeated for another 14 doses, as tolerated. The effective dose of MINC-IL-12 in the same dosage range is administered 1 dose a day for 3 days of 9 doses total.

The present invention is useful in human medicine and in veterinary medicine. The present invention is useful in treating human and non-human animals. For example, the present invention is useful in treating a mammal subject, such as humans, horses, pigs, cats, and dogs.

The following examples further illustrate the present invention. These examples are intended merely to be illustrative of the present invention and are not to be construed as being limiting.

### EXAMPLES

Table 1 lists the suppliers for raw materials used in the examples.

TABLE 1

| MATERIAL | SUPPLIER |
|---|---|
| IFN-α | PharmaEssentia |
| Trastuzumab | Eirgenix |
| IL-12 | Biolegend |
| Anti-CD3 monoclonal antibody | Biolegend |
| Anti-CD71 monoclonal antibody | Biolegend |
| Anti-EGFR monoclonal antibody | Biolegend |
| Anti-Tau monoclonal antibody | Biolegend |
| Anti-VEGF monoclonal antibody | Biolegend |
| IFN-γ | Biolegend |
| IL-2 | Biolegend |
| IL-6 | Biolegend |
| IL-15 | Biolegend |
| IL-21 | Biolegend |
| TRAIL | Biolegend |
| Bovine serum albumin | Apolo biochemical |
| EGCG | Biosynth Carbosynth |
| (−)-epigallocatechin gallate | |
| PEG-CHO (MW 5000) | NOF Corporation |
| ME-050AL | |
| DMSO | Macron |
| Centrifugal filter unit | GE Healthcare (Vivaspin) |
| (10KDa MWCO cut-off) | |
| 13 mm, PVDF membrane, 0.22 μm | JET BIOFIL |
| filter (with an average pore | |
| size of 0.22 μm) | |
| 17β-estradiol pellet | Innovative Research of America |
| Matrigel | BD Bioscience |

OEGCG and PEG-dEGCG were synthesized according to WO2006/12400, [0099] and [00102].

### Example 1. Method for Preparing MINC-Trastuzumab Nanoparticles

MINC (Multi-target Immune Nanocarrier Combination)-Trastuzumab Nanoparticles were prepared according to the following protocol:
1. Incubate 5 mg trastuzumab (34.4 nmol) solution in 10 ml PBS, at 37° C. for 1 hour.
2. Add 16.7 μl of OEGCG (30 mM in DMSO, 501 nmol).
3. Add 65 μl of PEG-EGCG (16 mM in DMSO).
4. Incubate the mixture at 25° C. for 3 hours.
5. Transfer the liquid into 10K MWCO centrifugal filter unit.
6. Centrifuge to reduce the liquid volume to 1 ml.
7. Add 9 ml, 0.9% NaCl and mix.
8. Repeat steps 6 and 7 for 3 additional times.
9. Filter through 0.22 μm membrane.
10. Transfer to cryotubes and stepwise freeze at 4° C. for 1 hour, −30° C. for 1 hour, then 80° C. overnight.
11. Lyophilize for 3 days.

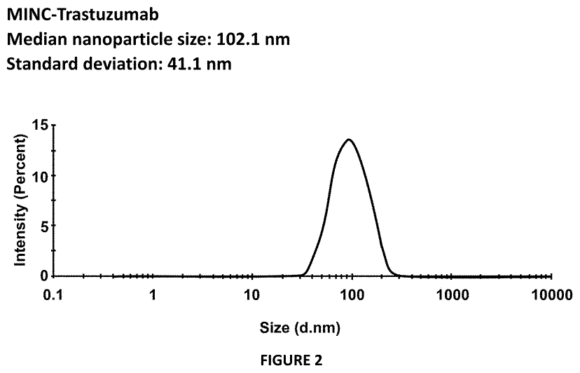

Nanoparticle size was measured by DLS (Malvern Zetasizer Nano ZS). Final product is shown in FIG. 2 with the median nanoparticle size of 102.1 nm. The standard deviation was 41.1 nm. The trastuzumab to OEGCG in molar ratio was 1:15. 100% of nanoparticles were distributed within 50 to 300 nm.

### Example 2. Method for Preparing MINC-IL-12 Nanoparticles

MINC-IL-12 Nanoparticles were prepared according to the following protocol:
1. Incubate 0.5 mg IL-12 (8.7 nmol) solution in 1 ml PBS, at 37° C. for 1 hour.
2. Add 5 μl of OEGCG (30 mM in DMSO, 150 nmol).
3. Add 10 μl of PEG-EGCG (16 mM in DMSO).
4. Incubate the mixture at 25° C. for 15 hours.
5. Transfer the liquid into 10K MWCO centrifugal filter unit.
6. Centrifuge to reduce the liquid volume to 0.1 ml.
7. Add 0.9 ml, 0.9% NaCl and mix.
8. Repeat steps 6 and 7 for 3 additional times.
9. Filter through 0.22 μm membrane.
10. Transfer to cryotubes and stepwise freeze at 4° C. for 1 hour, −30° C. for 1 hour, then −80° C. overnight.
11. Lyophilize for 3 days.

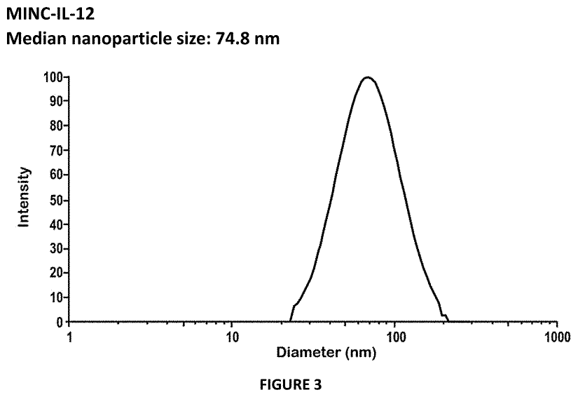

Nanoparticle size was measured by DLS (Malvern Zetasizer Nano ZS). Final product is shown in FIG. 3 with the median nanoparticle size of 74.8 nm. The IL-12 to OEGCG in molar ratio was 1:17. 90% of the particles have a desired molecular size of 50-300 nm.

### Example 3. Method for Preparing MINC-Anti-CD3 Nanoparticles

MINC-Anti-CD3 Nanoparticles were prepared according to the following protocol:
1. Incubate 0.5 mg Anti-CD3 (3.43 nmol) in 1 ml PBS, at 37° C. for 1.0 hour.
2. Add 6.7 μl of OEGCG (30 mM in DMSO, 201 nmol).
3. Add 26.6 μl of PEG-EGCG (16 mM in DMSO).
4. Incubate the mixture at 25° C. for 3 hours.
5. Transfer the liquid into 10K MWCO centrifugal filter unit.
6. Centrifuge to reduce the liquid to 0.1 ml.
7. Dissolve into 0.9 ml, 0.9% NaCl.
8. Repeat steps 6 and 7 for 3 additional times.
9. Filter with 0.22 μm membrane for 3 times.
10. Transfer to cryotubes and stepwise freeze at 4° C. for 1 hour, −30° C. for 1 hour, then 80° C. overnight.
11. Lyophilize for 3 days.

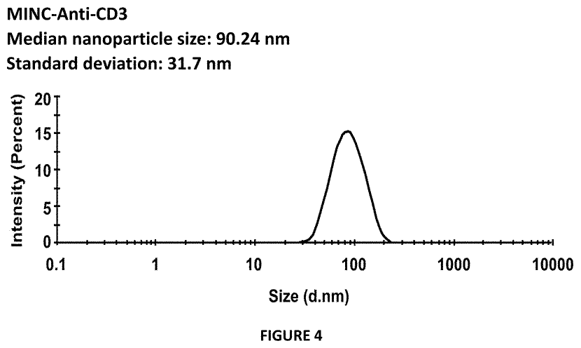

Nanoparticle size was measured by DLS (Malvern Zetasizer Nano ZS). Final product is shown in FIG. 4 with the median nanoparticle size of 90.24 nm. The standard deviation was 31.7 nm. The Anti-CD3 to OEGCG in molar ratio was 1:59. More than 95% of the nanoparticles were distributed within 50-300 nm.

### Example 4. Method for Preparing MINC-INFα Nanoparticles

MINC-INFα Nanoparticles were prepared according to the following protocol:
1. Incubate 0.067 mg (3.48 nmol) of IFN-α in 1 ml ix PBS, at 37° C. for 80 minutes.
2. Add 1.67 μl of OEGCG (30 mM in DMSO, 50.1 nmol).
3. Add 6.5 μl of PEG-EGCG (16 mM in DMSO).
4. Incubate the mixture at 25° C. for 3 hours.
5. Transfer the liquid into 10K MWCO centrifugal filter unit.
6. Centrifuge to reduce the liquid to 0.1 ml.
7. Dissolve into 0.9 ml, 0.9% NaCl.
8. Repeat steps 6 and 7 for 3 additional times.
9. Filter with 0.22 μm membrane.
10. Transfer to cryotubes and stepwise freeze at 4° C. for 1 hour, −30° C. for 1 hour, then 80° C. overnight.
11. Lyophilize for 3 days.

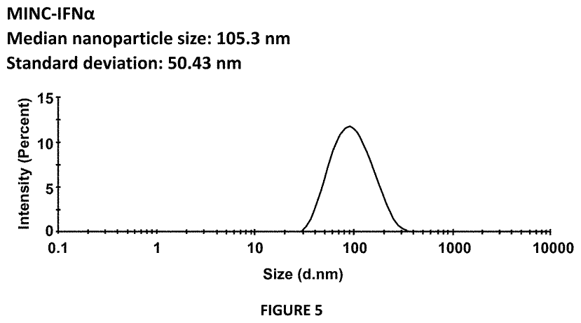

Nanoparticle size was measured by DLS (Malvern Zetasizer Nano ZS). Final product is shown in FIG. 5 with the median nanoparticle size of 105.3 nm. The standard deviation was 50.43 nm. The INFα to OEGCG in molar ratio was 1:14. More than 80% of nanoparticles were distributed within 50 to 300 nm.

### Example 5. Comparison of MINC-Trastuzumab Nanoparticles Prepared With Different

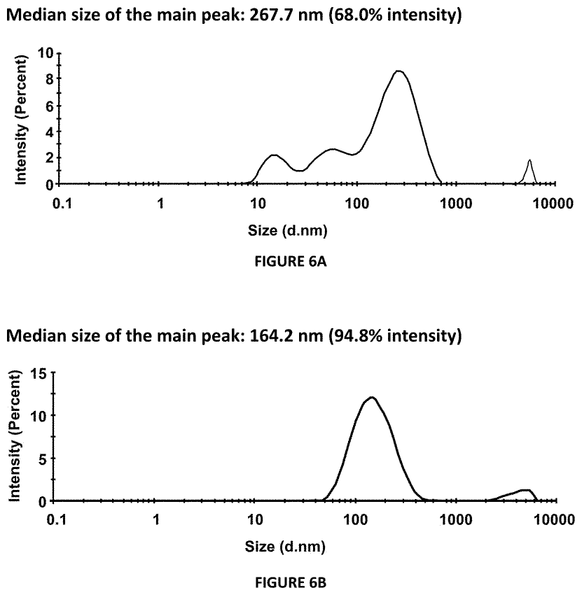

Trastuzumab to OEGCG Molar Ratios MINC-Trastuzumab nanoparticles (FIG. 6B) were prepared according to Example 1.

MINC-Trastuzumab nanoparticles (FIG. 6A) were prepared according to Example 1, except in step 2, which added 3.34 μl of OEGCG (30 mM in DMSO, 100.2 nmole). FIG. 6B was added 16.7 μl of OEGCG (30 mM in DMSO, 501 nmole). The nanoparticle size distribution of FIGS. 6A and 6B are very different. The results showed that when the protein to OEGCG (EGCG unit) molar ratio was changed from 1:15 (FIG. 6B) to 1:3 (FIG. 6A), the nanoparticle size distribution changed to multiple peaks. In addition, the median size of the main peak (Peak 1) in FIG. 6B is 164.2 nm (94.8% intensity), whereas the median size of the main peak (peak 1) in FIG. 6A is 267.7 nm (68.0% intensity).

### Example 6. Comparison of MINC-Trastuzumab Nanoparticles Prepared With or without 10K Molecular Weight Ultrafiltration

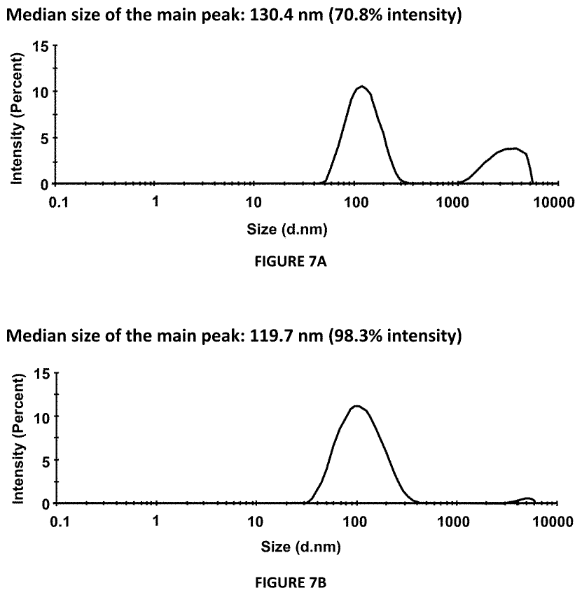

MINC-Trastuzumab Nanoparticles (FIG. 7B) were prepared according to Example 1. MINC-Trastuzumab Nanoparticles (FIG. 7A) were prepared according to Example 1, except without 10KDa MWCO centrifugal filtration (steps 5-8). The results show that without 10KDa MWCO ultrafiltration, the nanoparticle size distribution changed to multiple peaks (FIG. 7A). In addition, the median size of the main peak (Peak 1) in FIG. 7B is 119.7 nm (98.3% intensity), whereas the median size of the main peak (Peak 1) in FIG. 7A is 130.4 nm (70.8% intensity).

### Example 7. Comparison of MINC-Trastuzumab Nanoparticles Prepared With or Without 0.22 μm Filtration

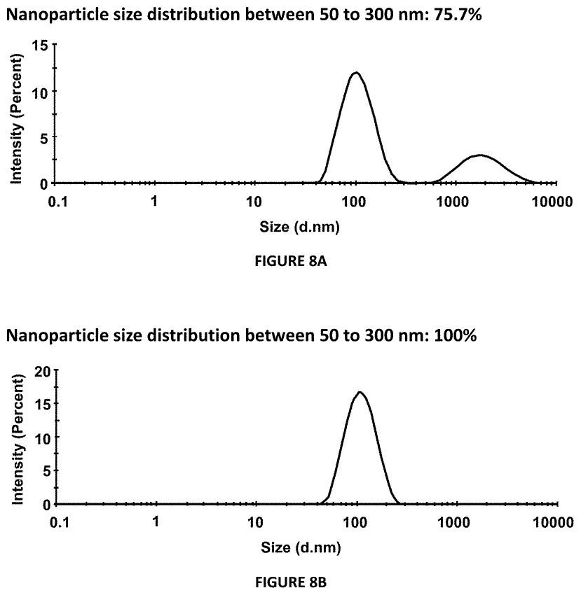

MINC-Trastuzumab nanoparticles (FIG. 8B) were prepared according to Example 1. Nanoparticles (FIG. 8A) were prepared according to Example 1, except without 0.22 μm filtration (step 9). DLS (Malvern Zetasizer Nano ZS) was used to measure the size of MINC-Trastuzumab Nanoparticles with 0.22 μm membrane filtration (FIG. 8B) and without 0.22 μm membrane filtration (FIG. 8A). With 0.22 μm filter (FIG. 8B), undesirable nanoparticles (larger than 300 nm) were removed, and the purity was increased. The nanoparticle size distribution between 50 to 300 nm was improved from 75.7% (FIG. 8A) to 100% (FIG. 8B).

### Example 8. Comparison of Trastuzumab Nanoparticles Prepared With Stepwise Freezing vs. Other Methods of Freezing for Lyophilization

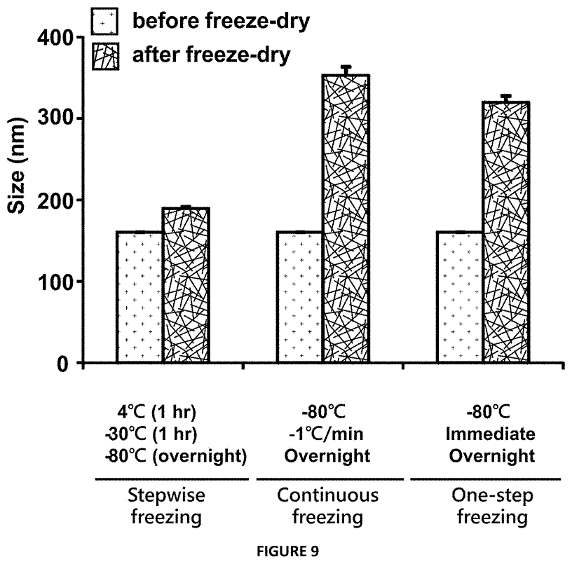

In FIG. 9, MINC-Trastuzumab nanoparticles were prepared either according to Example 1, or with a different step of step 10. DLS (Malvern Zetasizer Nano ZS) was used to measure the size of MINC-Trastuzumab nanoparticles under (i) stepwise freezing, (ii) continuous freezing at −1° C./min to −80° C., and (iii) one-step (immediate freezing) at −80° C. in step 10 (see FIG. 9). In the continuous freezing and one-step freezing procedure, the samples were put into a −80° C. with or without freezing container (−1° C./min, Thermo Scientific). In the stepwise freezing, samples were put into 4° C. for 1 hour, −30° C. for 1 hour, and then transferred to −80° C. for overnight. The results show that stepwise freezing procedure (4° C., −30° C., −80° C.) retained the size of MINC-Trastuzumab while continuous freezing or immediate freezing significantly increased the size of MINC-Trastuzumab after lyophilization (FIG. 9).

### Example 9. Biological Activity of MINC-Trastuzumab Nanoparticles

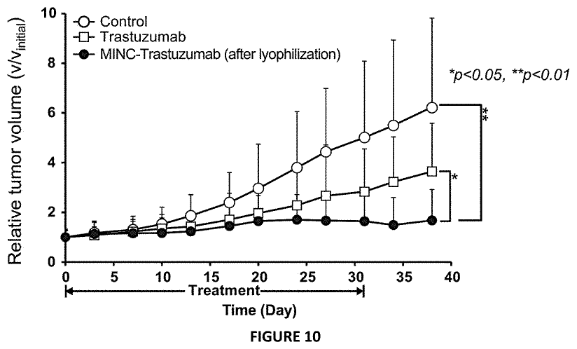

MINC-Trastuzumab Nanoparticles were prepared according to Example 1. In FIG. 10, lyophilized MINC-Trastuzumab was reconstituted with phosphate-buffered saline (PBS) and used for in vivo tumor suppression assay in BT474-xenografted mouse model (n=7-10 for each group). For the BT474-xenografted mouse model, Balb/nude mice were subcutaneously injected with 170-estradiol pellet (0.72 mg, 60 day-release). The following day, a suspension of 8×10⁶ BT474 cells (in 100 μL Matrigel) were subcutaneously injected in each mouse. The tumors were allowed to grow for 2 weeks before any treatment. Two weeks after tumor injection, mice were intravenously injected with drugs twice per week for 4 weeks. The mice were divided into 3 treatment groups with Trastuzumab (2.5 mg/kg), MINC-Trastuzumab (equivalent to 2.5 mg/kg Trastuzumab in the beginning of formulation), and PBS as vehicle control, respectively. The tumor size was measured by length (1) and width (w) using caliper. Tumor volume (V) was calculated as V=1w²/2 twice per week and normalized to the tumor size at first measurement as described (W.P. McGuire et al., N. Engl. J Med. 1996, 344(1), 1268). The results showed that the anticancer activity was successfully retained during the full preparation process, and the activity of MINC-Trastuzumab nanoparticles is better than Trastuzumab.

### Example 10. Method for Preparing MINC-Anti-CD71 Nanoparticles

MINC-Anti-CD71 Nanoparticles were prepared according to the following protocol:
1. Incubate 0.0625 mg Anti-CD71 (0.42 pmol) in 0.5 ml PBS, at 37° C. for 1.0 hour.
2. Add 5 μl of OEGCG (1.25 mM in DMSO, 6.25 pmol).
3. Add 10 μl of PEG-EGCG (1.1 mM in DMSO).
4. Incubate the mixture at 25° C. for 3 hours.
5. Transfer the liquid into 10K MWCO centrifugal filter unit.
6. Centrifuge to reduce the liquid to 0.1 ml.
7. Dissolve into 0.9 ml, 0.9% NaCl.
8. Repeat steps 6 and 7 for 1 additional time.
9. Filter with 0.22 μm membrane.
10. Transfer to cryotubes and stepwise freeze at 4° C. for 1 hour, −30° C. for 1 hour, then −80° C. overnight.
11. Lyophilize for 3 days.

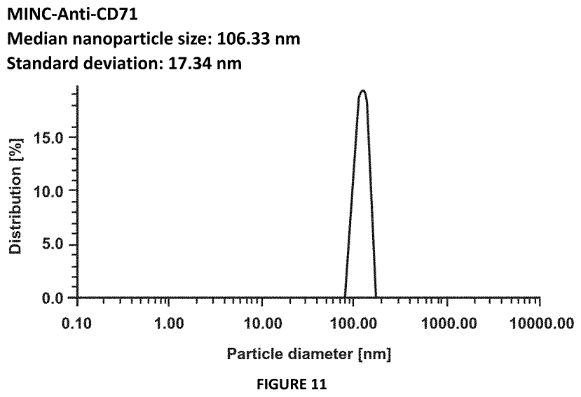

Nanoparticle size was measured by DLS (Anton Paar, Litesizer 500). Final product is shown in FIG. 11 with the median nanoparticle size of 106.33 nm. The standard deviation was 17.34 nm. The Anti-CD71 to OEGCG in molar ratio was 1:15. More than 95% of the nanoparticles were distributed within 50-300 nm.

### Example 11. Method for Preparing MINC-Anti-EGFR Nanoparticles

MINC-Anti-EGFR Nanoparticles were prepared according to the following protocol:
1. Incubate 0.0625 mg Anti-EGFR (0.42 pmol) in 0.5 ml PBS, at 37° C. for 1 hour.
2. Add 5 μl of OEGCG (5 mM in DMSO, 25 pmol).
3. Add 10 μl of PEG-EGCG (4.4 mM in DMSO).
4. Incubate the mixture at 25° C. for 3 hours.
5. Transfer the liquid into 10K MWCO centrifugal filter unit.
6. Centrifuge to reduce the liquid to 0.1 ml.
7. Dissolve into 0.9 ml, 0.9% NaCl.
8. Repeat steps 6 and 7 for 1 additional time.
9. Filter with 0.22 μm membrane.
10. Transfer to cryotubes and stepwise freeze at 4° C. for 1 hour, −30° C. for 1 hour, then −80° C. overnight.
11. Lyophilize for 3 days.

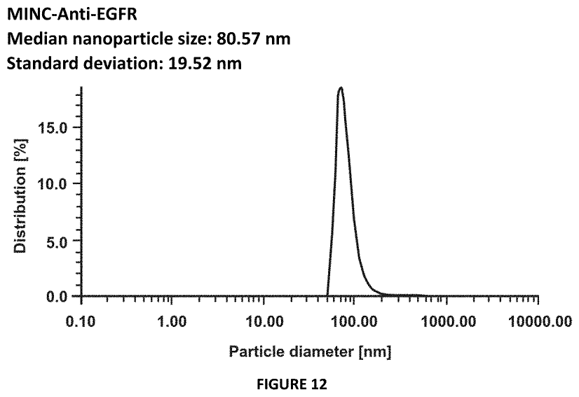

Nanoparticle size was measured by DLS (Anton Paar, Litesizer 500). Final product is shown in FIG. 12 with the median nanoparticle size of 80.57 nm. The standard deviation was 19.52 nm. The Anti-EGFR to OEGCG in molar ratio was 1:60. More than 95% of the nanoparticles were distributed within 50-300 nm.

### Example 12. Method for Preparing MINC-Anti-Tau Nanoparticles

MINC-Anti-Tau Nanoparticles were prepared according to the following protocol:
1. Incubate 0.0625 mg Anti-Tau (0.42 pmol) in 0.5 ml PBS, at 37° C. for 1.0 hour.
2. Add 5 μl of OEGCG (5 mM in DMSO, 25 pmol).
3. Add 10 μl of PEG-EGCG (4.4 mM in DMSO).
4. Incubate the mixture at 25° C. for 3 hours.
5. Transfer the liquid into 10K MWCO centrifugal filter unit.
6. Centrifuge to reduce the liquid to 0.1 ml.
7. Dissolve into 0.9 ml, 0.9% NaCl.
8. Repeat steps 6 and 7 for 1 additional time.
9. Filter with 0.22 μm membrane.
10. Transfer to cryotubes and stepwise freeze at 4° C. for 1 hour, −30° C. for 1 hour, then −80° C. overnight.
11. Lyophilize for 3 days.

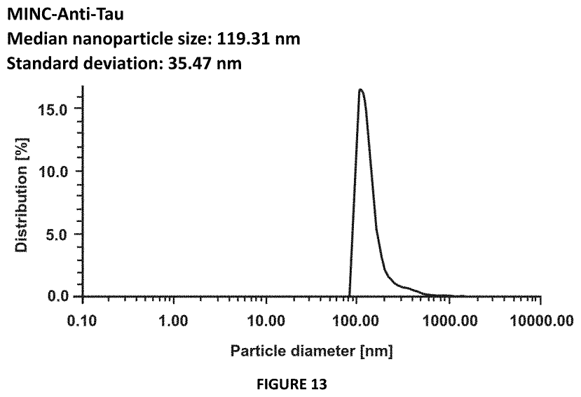

Nanoparticle size was measured by DLS (Anton Paar, Litesizer 500). Final product is shown in FIG. 13 with the median nanoparticle size of 119.31 nm. The standard deviation was 35.47 nm. The Anti-Tau to OEGCG in molar ratio was 1:60. More than 95% of the nanoparticles were distributed within 50-300 nm.

### Example 13. Method for Preparing MINC-Anti-VEGF Nanoparticles

MINC-Anti-VEGF Nanoparticles were prepared according to the following protocol:
1. Incubate 0.0625 mg Anti-VEGF (0.42 pmol) in 0.5 ml PBS, at 37° C. for 1 hour.
2. Add 5 μl of OEGCG (1.25 mM in DMSO, 6.25 pmol).
3. Add 10 μl of PEG-EGCG (1.1 mM in DMSO).
4. Incubate the mixture at 25° C. for 3 hours.
5. Transfer the liquid into 10K MWCO centrifugal filter unit.
6. Centrifuge to reduce the liquid to 0.1 ml.
7. Dissolve into 0.9 ml, 0.9% NaCl.
8. Repeat steps 6 and 7 for 1 additional time.
9. Filter with 0.22 μm membrane.
10. Transfer to cryotubes and stepwise freeze at 4° C. for 1 hour, −30° C. for 1 hour, then −80° C. overnight.
11. Lyophilize for 3 days.

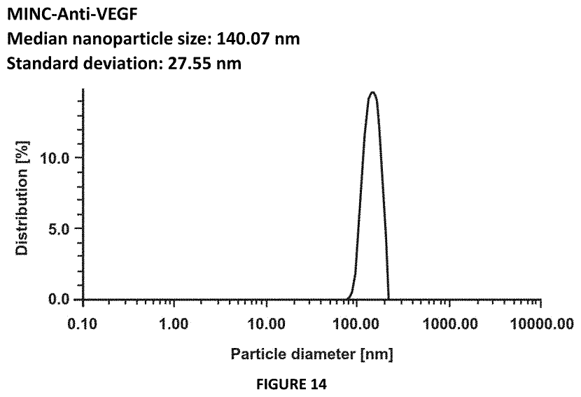

Nanoparticle size was measured by DLS (Anton Paar, Litesizer 500). Final product is shown in FIG. 14 with the median nanoparticle size of 140.07 nm. The standard deviation was 27.55 nm. The Anti-VEGF to OEGCG in molar ratio was 1:15. More than 95% of the nanoparticles were distributed within 50-300 nm.

### Example 14. Method for Preparing MINC-IFN-7 Nanoparticles

MINC-IFN-γ Nanoparticles were prepared according to the following protocol:
1. Incubate 0.0625 mg IFN-7 (3.98 pmol) in 0.5 ml PBS, at 37° C. for 1.0 hour.
2. Add 5 μl of OEGCG (5 mM in DMSO, 25 pmol).
3. Add 10 μl of PEG-EGCG (4.4 mM in DMSO).
4. Incubate the mixture at 25° C. for 3 hours.
5. Transfer the liquid into 10K MWCO centrifugal filter unit.
6. Centrifuge to reduce the liquid to 0.1 ml.
7. Dissolve into 0.9 ml, 0.9% NaCl.
8. Repeat steps 6 and 7 for 1 additional time.
9. Filter with 0.22 μm membrane.
10. Transfer to cryotubes and stepwise freeze at 4° C. for 1 hour, −30° C. for 1 hour, then −80° C. overnight.
11. Lyophilize for 3 days.

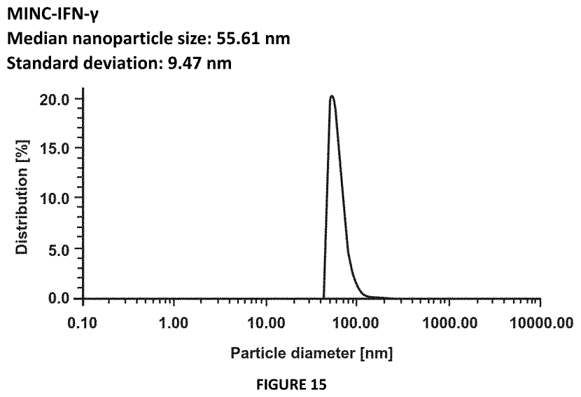

Nanoparticle size was measured by DLS (Anton Paar, Litesizer 500). Final product is shown in FIG. 15 with the median nanoparticle size of 55.61 nm. The standard deviation was 9.47 nm. The IFN-7 to OEGCG in molar ratio was 1:6. More than 85% of the nanoparticles were distributed within 50-300 nm.

### Example 15. Method for Preparing MINC-IL-2 Nanoparticles

MINC-IL-2 Nanoparticles were prepared according to the following protocol:
1. Incubate 0.0625 mg IL-2 (4.06 pmol) in 0.5 ml PBS, at 37° C. for 1.0 hour.
2. Add 5 μl of OEGCG (5 mM in DMSO, 25 pmol).
3. Add 10 μl of PEG-EGCG (4.4 mM in DMSO).
4. Incubate the mixture at 25° C. for 3 hours.
5. Transfer the liquid into 10K MWCO centrifugal filter unit.
6. Centrifuge to reduce the liquid to 0.1 ml.
7. Dissolve into 0.9 ml, 0.9% NaCl.
8. Repeat steps 6 and 7 for 1 additional time.
9. Filter with 0.22 μm membrane.
10. Transfer to cryotubes and stepwise freeze at 4° C. for 1 hour, −30° C. for 1 hour, then −80° C. overnight.
11. Lyophilize for 3 days.

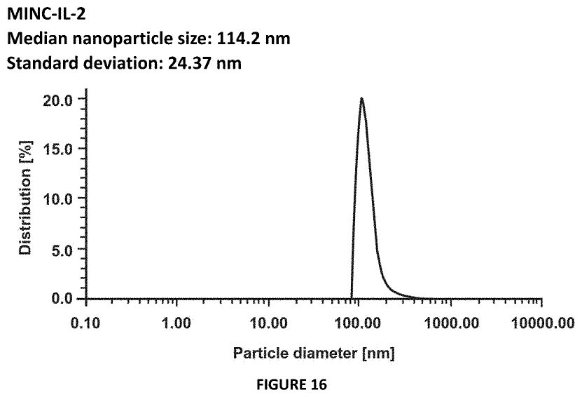

Nanoparticle size was measured by DLS (Anton Paar, Litesizer 500). Final product is shown in FIG. 16 with the median nanoparticle size of 114.2 nm. The standard deviation was 24.37 nm. The IL-2 to OEGCG in molar ratio was 1:6. More than 95% of the nanoparticles were distributed within 50-300 nm.

### Example 16. Method for Preparing MINC-IL-6 Nanoparticles

MINC-IL-6 Nanoparticles were prepared according to the following protocol:
1. Incubate 0.0625 mg IL-6 (2.85 pmol) in 0.5 ml PBS, at 37° C. for 1.0 hour.
2. Add 5 μl of OEGCG (5 mM in DMSO, 25 pmol).
3. Add 10 μl of PEG-EGCG (4.4 mM in DMSO).
4. Incubate the mixture at 25° C. for 3 hours.
5. Transfer the liquid into 10K MWCO centrifugal filter unit.
6. Centrifuge to reduce the liquid to 0.1 ml.
7. Dissolve into 0.9 ml, 0.9% NaCl.
8. Repeat steps 6 and 7 for 1 additional time.
9. Filter with 0.22 μm membrane.
10. Transfer to cryotubes and stepwise freeze at 4° C. for 1 hour, −30° C. for 1 hour, then −80° C. overnight.
11. Lyophilize for 3 days.

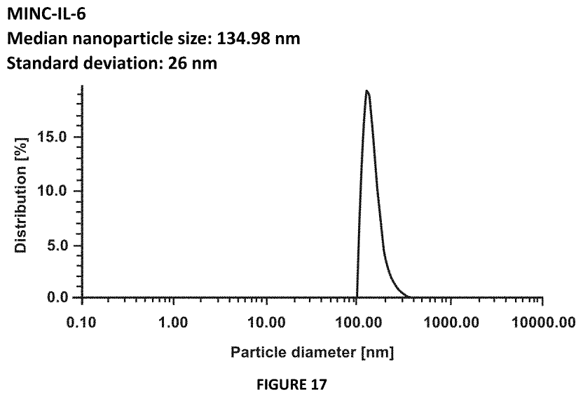

Nanoparticle size was measured by DLS (Anton Paar, Litesizer 500). Final product is shown in FIG. 17 with the median nanoparticle size of 134.98 nm. The standard deviation was 26 nm. The IL-6 to OEGCG in molar ratio was 1:9. More than 95% of the nanoparticles were distributed within 50-300 nm.

### Example 17. Method for Preparing MINC-IL-15 Nanoparticles

MINC-IL-15 Nanoparticles were prepared according to the following protocol:
1. Incubate 0.0625 mg IL-15 (4.66 pmol) in 0.5 ml PBS, at 37° C. for 1.0 hour.
2. Add 5 μl of OEGCG (5 mM in DMSO, 25 pmol).
3. Add 10 μl of PEG-EGCG (4.4 mM in DMSO).
4. Incubate the mixture at 25° C. for 3 hours.
5. Transfer the liquid into 10K MWCO centrifugal filter unit.
6. Centrifuge to reduce the liquid to 0.1 ml.
7. Dissolve into 0.9 ml, 0.9% NaCl.
8. Repeat steps 6 and 7 for 1 additional time.
9. Filter with 0.22 μm membrane.
10. Transfer to cryotubes and stepwise freeze at 4° C. for 1 hour, −30° C. for 1 hour, then −80° C. overnight.
11. Lyophilize for 3 days.

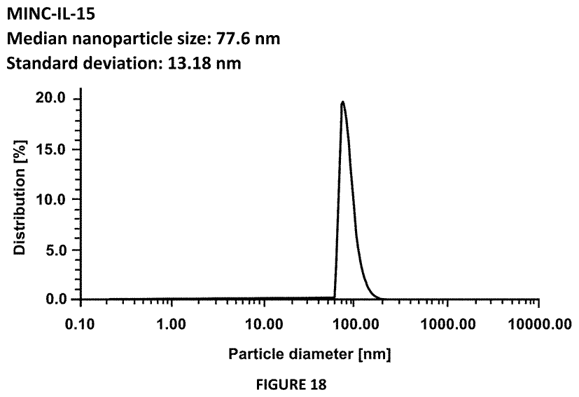

Nanoparticle size was measured by DLS (Anton Paar, Litesizer 500). Final product is shown in FIG. 18 with the median nanoparticle size of 77.6 nm. The standard deviation was 13.18 nm. The IL-15 to OEGCG in molar ratio was 1:5. More than 95% of the nanoparticles were distributed within 50-300 nm.

### Example 18. Method for Preparing MINC-IL-21 Nanoparticles

MINC-IL-21 Nanoparticles were prepared according to the following protocol:
1. Incubate 0.0625 mg IL-21 (4.40 pmol) in 0.5 ml PBS, at 37° C. for 1.0 hour.
2. Add 5 μl of OEGCG (5 mM in DMSO, 25 pmol).
3. Add 10 μl of PEG-EGCG (4.4 mM in DMSO).
4. Incubate the mixture at 25° C. for 3 hours.
5. Transfer the liquid into 10K MWCO centrifugal filter unit.
6. Centrifuge to reduce the liquid to 0.1 ml.
7. Dissolve into 0.9 ml, 0.9% NaCl.
8. Repeat steps 6 and 7 for 1 additional time.
9. Filter with 0.22 μm membrane.
10. Transfer to cryotubes and stepwise freeze at 4° C. for 1 hour, −30° C. for 1 hour, then −80° C. overnight.
11. Lyophilize for 3 days.

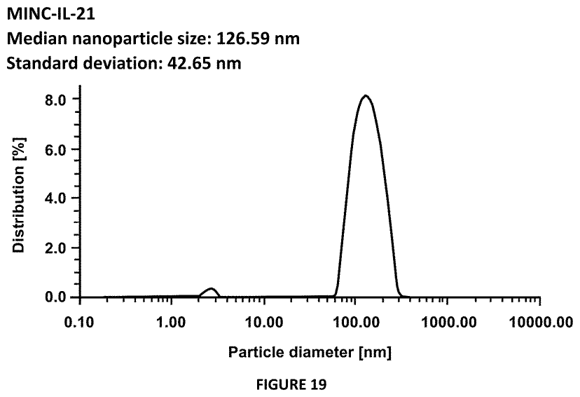

Nanoparticle size was measured by DLS (Anton Paar, Litesizer 500). Final product is shown in FIG. 19 with the median nanoparticle size of 126.59 nm. The standard deviation was 42.65 nm. The IL-21 to OEGCG in molar ratio was 1:6. More than 95% of the nanoparticles were distributed within 50-300 nm.

### Example 19. Method for Preparing MINC-TRAIL Nanoparticles

MINC-TRAIL Nanoparticles were prepared according to the following protocol:
1. Incubate 0.0625 mg TRAIL (2.75 pmol) in 0.5 ml PBS, at 37° C. for 1.0 hour.
2. Add 5 μl of OEGCG (5 mM in DMSO, 25 pmol).
3. Add 10 μl of PEG-EGCG (4.4 mM in DMSO).
4. Incubate the mixture at 25° C. for 3 hours.
5. Transfer the liquid into 10K MWCO centrifugal filter unit.
6. Centrifuge to reduce the liquid to 0.1 ml.
7. Dissolve into 0.9 ml, 0.9% NaCl.
8. Repeat steps 6 and 7 for 1 additional time.
9. Filter with 0.22 μm membrane.
10. Transfer to cryotubes and stepwise freeze at 4° C. for 1 hour, −30° C. for 1 hour, then −80° C. overnight.
11. Lyophilize for 3 days.

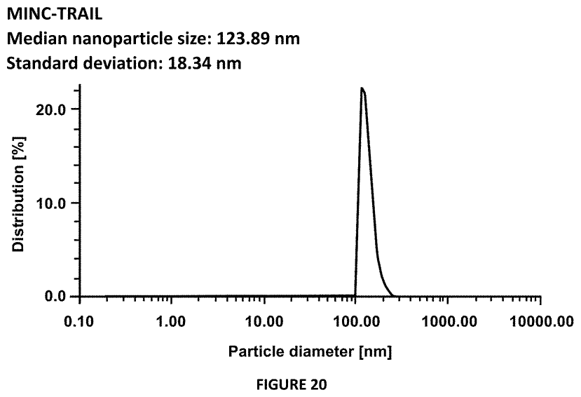

Nanoparticle size was measured by DLS (Anton Paar, Litesizer 500). Final product is shown in FIG. 20 with the median nanoparticle size of 123.89 nm. The standard deviation was 18.34 nm. The TRAIL to OEGCG in molar ratio was 1:9. More than 95% of the nanoparticles were distributed within 50-300 nm.

### Example 20. Method for Preparing MINC-BSA Nanoparticles

MINC-BSA Nanoparticles were prepared according to the following protocol:
1. Incubate 0.5 mg BSA (0.42 pmol) in 1 ml PBS, at 37° C. for 1.0 hour.
2. Add 5 μl of OEGCG (5 mM in DMSO, 25 pmol).
3. Add 10 μl of PEG-EGCG (4.4 mM in DMSO).
4. Incubate the mixture at 25° C. for 3 hours.
5. Transfer the liquid into 10K MWCO centrifugal filter unit.
6. Centrifuge to reduce the liquid to 0.1 ml.
7. Dissolve into 0.9 ml, 0.9% NaCl.
8. Repeat steps 6 and 7 for 1 additional time.
9. Filter with 0.22 μm membrane.
10. Transfer to cryotubes and stepwise freeze at 4° C. for 1 hour, −30° C. for 1 hour, then −80° C. overnight.
11. Lyophilize for 3 days.

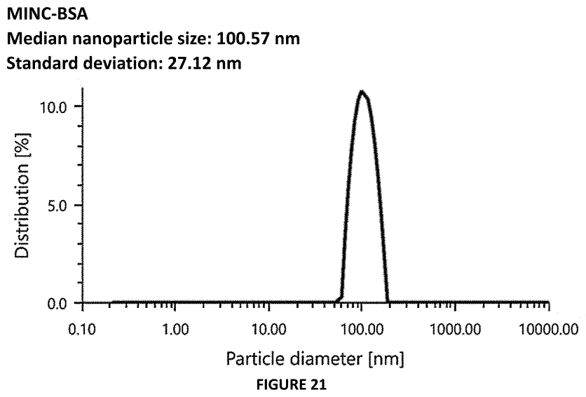

Nanoparticle size was measured by DLS (Anton Paar, Litesizer 500). Final product is shown in FIG. 21 with the median nanoparticle size of 100.57 nm. The standard deviation was 27.12 nm. The BSA to OEGCG in molar ratio was 1:19. More than 95% of the nanoparticles were distributed within 50-300 nm.

The invention, and the manner and process of making and using it, are now described in such full, clear, concise, and exact terms as to enable any person skilled in the art to which it pertains, to make and use the same. It is to be understood that the foregoing describes preferred embodiments of the present invention and that modifications may be made therein without departing from the scope of the present invention as set forth in the claims. To particularly point out and distinctly claim the subject matter regarded as the invention, the following claims conclude this specification.

What is claimed is:

1. A nanoparticle composition comprising nanoparticles having: (a) an inner core comprising oligomeric (−)-epigallocatechin gallate (OEGCG), (b) an outer core comprising a polyethylene glycol-epigallocatechin gallate conjugate (PEG-EGCG), and (c) a drug molecule encapsulated in the inner core; wherein the drug molecule is an antibody or a cytokine, at least 70% of the nanoparticles have a diameter between 50-300 nm, and the size distribution of the nanoparticles only has one major peak containing more than 90% of all the particles.

2. The nanoparticle composition according to claim 1, wherein at least 80% of the nanoparticles have a diameter between 50-300 nm.

3. The nanoparticle composition according to claim 1, wherein at least 90% of the nanoparticles have a diameter between 50-300 nm.

4. The nanoparticle composition according to claim 1, wherein the median diameter of the nanoparticles is about 50-250 nm.

5. The nanoparticle composition according to claim 1, wherein the median diameter of the nanoparticles is about 50-200 nm.

6. The nanoparticle composition according to claim 1, wherein the size distribution of the nanoparticles has only one major peak containing more than 95% of all the particles.

7. The nanoparticle composition according to claim 1, wherein the antibody is a monoclonal antibody.

8. The nanoparticle composition according to claim 1, wherein the antibody is anti-HER2, anti-CD71, anti-EGFR, anti-VEGF, or anti-Tau.

9. The nanoparticle composition according to claim 1, wherein the cytokine is an interferon, an interleukin, a lymphokine, or a tumor necrosis factor.

10. The nanoparticle composition according to claim 1, wherein the cytokine is IL-12, IL-2, IL-6, IL-15, IL-21, IFN-α, IFN-7, or TRAIL.

11. The process for preparing the nanoparticle composition of claim 1, comprising the steps of:
(a) mixing a drug protein molecule with OEGCG and PEG-EGCG in an aqueous solution,
(b) filtering the mixture through a membrane with a molecular weight cut-off of 8,000-300,000 daltons to remove small molecular weight molecules and retain large molecular weight molecules, and
(c) filtering the large molecular weight molecules through 0.2-0.3 μm membrane and collecting the filtrate.

12. The process according to claim 11, further comprising a step (d) after step (c):
(d) lyophilizing the filtrate by stepwise freezing at (i) about 0-5° C., (ii) about −20 to −30° C., and (iii) at about −60 to −100° C., and then drying.

13. The process according to claim 11, wherein the molar ratio of the EGCG in OEGCG to the drug molecule is 5-100 to 1.

14. The process according to claim 13, wherein the molar ratio of the EGCG in OEGCG to the drug molecule is 10-50 to 1.

15. The process according to claim 12, wherein the molar ratio of the EGCG in OEGCG to the drug molecule is 5-100 to 1.

16. The process according to claim 11, wherein the molecular weight cut-off in step (b) is 8,000-12,000 daltons.

17. The process according to claim 12, wherein the molecular weight cut-off in step (b) is 8,000-12,000 daltons.

18. The process according to claim 11, wherein the steps (b) and (c) are repeated 1, 2, 3, or 4 times before step (d).

19. The process according to claim 12, wherein the stepwise freezing is performed at (i) about 0-5° C. for at least 1 hour, (ii) about −20° C. to −30° C., for at least 1 hour, and (iii) at −60° C. to −100° C. for at least 2 hours.

20. A method for treating cancer, comprising the step of administering an effective amount of the nanoparticle composition of claim 1 to a subject in need thereof.
